(19) United States
(12) Patent Application Publication
(10) Pub. No.: US 2002/0082933 A1
(43) Pub. Date: Jun. 27, 2002

(54) ECG investment planning method and apparatus

(76) Inventor: Richard F. Wehrhahn, Greenfield, WI (US)

Correspondence Address: Patrick S. Yoder, Suite 330, 7915 FM 1960 West, Houston, TX, 77070, US

(21) Appl. No.: 09/746,951

(22) Filed: Dec. 22, 2000

(51) Int. Cl. G06F 17/60; A61B 5/00

(52) U.S. Cl. 705/26; 600/300

(57) ABSTRACT

A system and method for providing a resting ECG system customer with a recommended resting ECG system. The recommended resting ECG system being established by a customer's responses to a plurality of questions provided to the customer over a network via a query page. The system having a product selector file and a product configuration file. The product selector file provides the query page with the plurality of questions to be answered by the customer. The product configuration file having a plurality of resting ECG systems associated with specific responses to the questions on the query page. A Java applet being used to compare the customer's responses to the plurality of questions to the information in the product configuration file. The product configuration file supplying a results page with the recommended system based on the customer's responses to the questions on the query page. The system providing the results page to the customer over the network.

### FIELD OF THE INVENTION

The present invention relates generally to a method and apparatus for providing purchasing information to a resting electrocardiograph (ECG) customer, and more particularly, to a method and apparatus to enable a resting ECG system supplier to provide a customer with a recommended resting ECG system.

### BACKGROUND OF THE INVENTION

ECG analysis is a well-established method for studying the function of the heart and identifying disorders of the heart. ECG analysis provides graphical information that can indicate abnormalities in the heart such as irregular heart rates or rhythms, or abnormal conduction pathways. Likewise, an ECG can indicate the location of damaged cardiac tissues if it exists. Perhaps best of all, the ECG allows a physician to diagnose abnormalities inexpensively, without using surgical procedures

An ECG is a graphic tracing of the variations and the electrical potential caused by the excitation of the heart muscle as detected at the body surface by leads of the ECG device. A normal cardiogram reflects the alternate contraction of the atria (the two upper chambers of the heart) and of the ventricles (the two lower chambers of the heart). An electrocardiogram is a scale or representation that shows deflections resulting from cardiac activity as changes in the magnitude of voltage and polarity over time and includes a P-Wave, a QRS complex, and a T-Wave. The P-Wave is due to atrial contraction and is known as the atrial complex. The Q, R S, and T deflections are all due to the action of the ventricles and are known as the ventricular complexes. These waves are analyzed using a set of rules and parameters to determine what is normal and what is not. Certain deviations are used to flag possible complications.

ECG examinations may be performed as the patient is experiencing different levels of heart activity. For example, ECG examinations may be performed while the patient has a high level of heart activity, such as during vigorous exercise, or at a lower level of activity, such as during a resting period. The different levels of heart activity provide different windows into the operation of the patient's heart. A resting ECG examination is an ECG test that is performed while the patient is in a resting condition, e.g., lying down. A resting ECG system is an ECG testing device that is designed to perform resting ECG examinations.

To purchase a resting ECG system, a customer may contact a supplier of resting ECG systems to request information regarding the supplier's resting ECG systems. Alternatively, a resting ECG system supplier may contact a customer in hopes of generating sales of resting ECG systems. Both parties in each of these cases may consume significant amounts of time attempting to elicit basic information from the other. For example, it may take a resting ECG system supplier a significant amount of time simply to establish the customer's basic resting ECG system needs or desires. Additionally, a customer may expend significant amounts of time obtaining basic information about the resting ECG systems available from the supplier. Indeed, the customer may consume time receiving information from a supplier about resting ECG systems that are simply not suited for the customer's needs or desires.

There is a need, therefore, for an improved technique for providing an resting ECG system customer with purchasing information regarding a supplier's resting ECG systems prior to contact between a sales representative of the supplier and the customer. There is a particular need for a system or method that provides a customer with information for a recommended resting ECG system and some basic information about the recommended resting ECG system.

### SUMMARY OF THE INVENTION

According to one aspect of the present invention, a system is featured. The system is coupled to a network to enable a resting ECG system supplier provide a customer with a recommended resting ECG system. The system comprises an application server, a comparison program and a server. The application server directs a query page to the customer via the network. The query page has a plurality of questions and a plurality of answer choices for each question. A customer's answers to the questions enable the system to determine a recommended resting ECG system. At least one question is designed to determine whether the customer desires software that will interpret results from a resting ECG examination to assist a physician to determine the patient's heart condition. The system also comprises a comparison program to receive a completed query page containing the customer's answers to the questions. The comparison program compares the customer's answers to information stored in the system that relates the customer's answers to specific resting ECG systems. The specific resting ECG that corresponds to a customer's answers being the recommended resting ECG system. The system also comprises a server to provide a results page to the customer via the network. The results page provides the customer with the recommended resting ECG system.

According to another aspect of the present invention, a computer system is featured. The computer system is coupled to a network to enable a resting electrocardiograph (ECG) system supplier to provide a customer with a recommended resting ECG system over the network. The computer system comprises an application server, a product selector file, a program, and a product configuration file. The application server is coupled to a network. The application server directs a customer from the network to files stored in the computer system. The product selector file is written in a markup language and stored in the computer system. The product selector file holds a plurality of questions designed to obtain data from a customer to determine a rested ECG system, from among a plurality of resting ECG systems, to recommend to the customer. The product selector file provides the plurality of questions to a query page for delivery to a customer. The program operates to determine a recommended resting ECG system for the customer by comparing data provided by the customer via the plurality of questions to resting ECG system data stored in the computer system. The product configuration file is written in a markup language and stored in the computer system. The product configuration file holds the resting ECG system data used by the program. The product configuration file also provides the recommended resting ECG system to a results page for delivery to the customer.

According to another aspect of the present invention, a method is featured of utilizing a computer system coupled to a network to assist a customer to configure a resting electrocardiograph (ECG) system. The method enables a supplier to recommend a resting ECG system to the customer from among a plurality of resting ECG systems, components and software. The method comprising the act of routing a request for assistance from a customer to a product selector file written in extensible markup language (XML). The product selector file fills a template with questions stored in the product selector file. The method also comprises the acts of delivering the template over the network to a customer and receiving a completed template from the customer. The method also comprises the act of determining a recommended resting ECG system configuration by comparing customer data derived from the completed template to supplier data stored in the computer system in a product configuration file written in XML. The product configuration file fills a results page with the recommended resting ECG system configuration for delivery to the customer over the network.

### BRIEF DESCRIPTION OF THE DRAWINGS

FIG. 1 is a visual representation of a person undergoing a resting ECG examination;

FIG. 2 is a list of questions for use in a product selector for a resting ECG system;

FIG. 3 is a template of resting ECG systems corresponding to predicted answers to the question of FIG. 2;

FIG. 4 is a diagrammatical representation of a computer system to enable a resting ECG system supplier to provide a customer with a recommendation for a resting ECG system;

FIG. 5 is a flowchart for a process whereby a resting ECG system supplier may provide a customer with a recommendation for a resting ECG system;

FIG. 6 is a representation of a page for an ECG system supplier, the page having a link to a resting ECG system product selector;

FIG. 7 is a representation of a query page for a resting ECG system product selector;

FIG. 8 is a representation of a help page for a resting ECG system product selector;

FIG. 9 is a representation of a results page featuring a recommended resting ECG system; and

FIG. 10 is a representation of a results page when the system cannot provide a recommended resting ECG system and directing the customer to contact a supplier's representative.

### DETAILED DESCRIPTION OF THE INVENTION

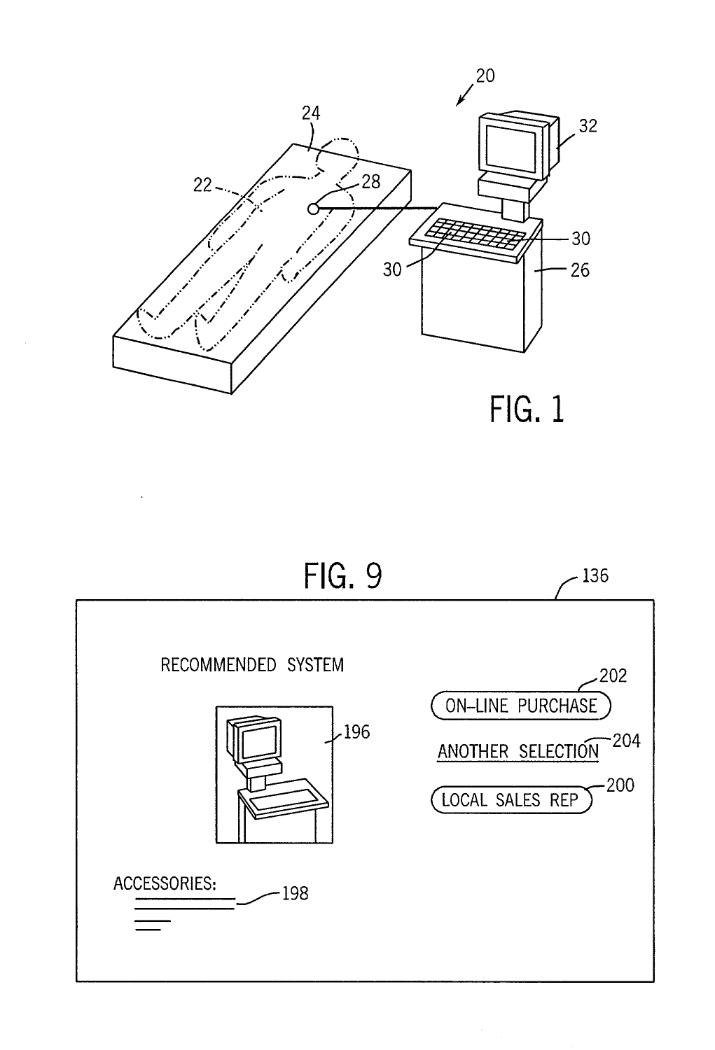

FIG. 1 illustrates a resting ECG system 20. To perform a resting ECG examination, a person 22 lies on a bed or examination table 24. An operator 30 controls the operation of the resting ECG system 20 from a console 26. Electrodes 28 are extended from the console 26 and placed at key locations on the body of the person 22. In the illustrated embodiment, the resting ECG system console 26 has a recorder 30 that records a graphical record of heart activity. The recorder 30 may print out the graphical record on paper or record the information digitally. In the illustrated embodiment, resting ECG system 20 also has a monitor 32 to provide visible information to the operator.

Resting ECG systems may be used in a variety of different clinical environments, such as large hospitals, small hospitals, clinics and private practices. Advanced resting ECG systems may be used in a large hospital where a large number of resting ECG examinations may be performed each week. Portable resting ECG systems may be utilized in a private practice or small clinic when the number of tests to be performed each week is relatively small, compared to a large hospital.

Additionally, resting ECG systems may utilize a variety of options. Some resting ECG systems are able to perform an interpretation of the patient's ECG. Additional software may be required to perform this function. Other diagnostic tools, such as P-wave signal averaging, also may be available. Additionally, different sized paper may be used in different resting ECG systems. Resting ECG systems may be connectable to an information network. Devices such as modems and faxes may be incorporated into the resting ECG systems to enable the resting ECG to communicate via the information network. Other options may be available, such as 15-lead electrodes and ACI-TIPI.

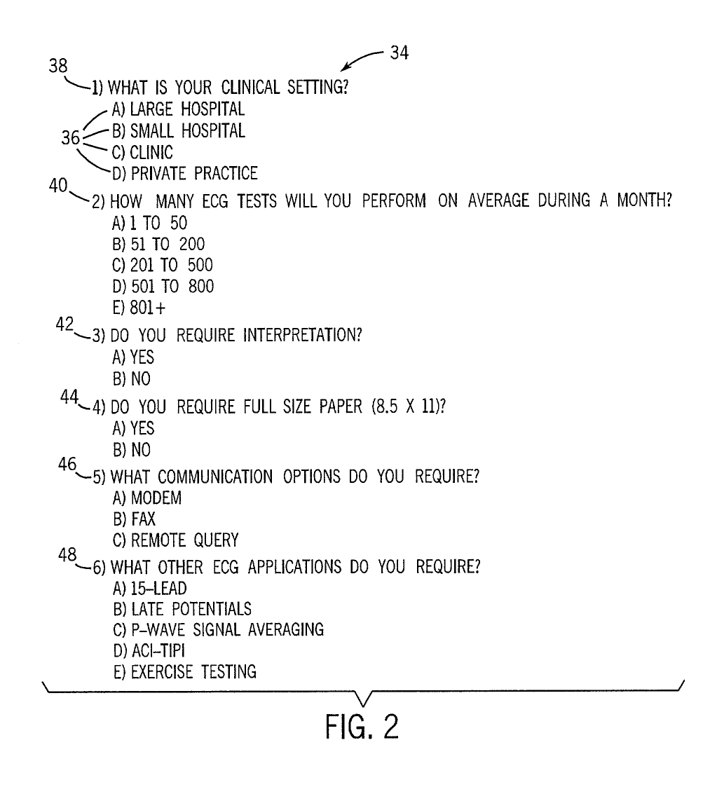

Referring generally to FIG. 2, an exemplary list 34 of questions that may be used to elicit information from a resting ECG customer is provided. The questions may be used to recommend a resting ECG system that meets the customer's needs or at least provide information to serve as a basis for discussion between a supplier and a customer. Each answer to each question is shown with an associated letter identifier 36. The questions and answers may vary from supplier to supplier.

In the list of questions, the first question 38 asks, “What is your clinical setting?” which is followed, in this embodiment, by four answer choices: “A. Large hospital”, “B. Small hospital”, “C. Clinic”, and “D. Private practice.” The answer may be used to gage the customer's requirement for a resting ECG system so that the supplier may recommend the most appropriate resting ECG system for the customer. Additionally, the supplier may have separate sales groups for different clinical settings. In that situation, the customer's response to the first question 38 may be used during discussions between the customer and the supplier to determine the appropriate sales group to do business with the customer.

Primarily, the questions are designed to elicit information from the customer so that a resting ECG system may be recommended to the customer. For example, the second question 40 asks, “How many ECG tests will you perform on average during a month?” The illustrated answer options for the second question 40 are: “A. 1 to 50”, “B. 51to 200”, “C. 201 to 500”, “D. 501 to 800”, and “E. 801+.” This information provides the supplier with the information needed to determine the appropriate size and capabilities of a basic resting ECG system. A resting ECG system that is only used to perform only a few resting ECG examinations a month need not be as robust as a resting ECG system for a customer that performs more than 800 examinations a month. This also gives an indication as to the price that a customer may be willing to pay for a resting ECG system. The more frequently the customer may use the resting ECG system, the more money they are probably willing to pay.

Recommending a resting ECG system may also encompass providing a customer with appropriate resting ECG system options. The resting ECG options may be components, software, consumables, etc. The questions may be designed to elicit information from the customer to determine which, if any, resting ECG system options the supplier should recommend to the customer. For example, the third question 42 asks the question “Do you require interpretation?” The illustrated answer choices for the third question 42 are: “A. Yes” or “B. No.” If the customer answers “A. Yes”, then interpretation software may be recommended to the customer. Interpretation software provides an operator with diagnostic tools to help in the analysis of the patient's cardiac condition. A customer requiring interpretation would most likely not be recommended a resting ECG system that did not have that capability.

Additional questions may serve a similar function. For example, the fourth question 44 asks, “Do you require full size paper?” The answer choices for the fourth question 44 are “A. Yes” or “B. No.” If the customer answers “A. Yes”, then a system that cannot provide that option will most likely not be recommended to the customer. Additionally, any options that may be needed to enable the customer to use full-sized paper may be recommended to the customer. The fifth question 46 asks, “What communication options do you require?” The illustrated answer choices are: “A. Modem”, “B. Fax”, and “C. Remote query.” A system that cannot provide the desired option would probably not be recommended to the customer. Similarly, the sixth question 48 asks, “What other ECG applications do you require?” The illustrated answer choices are: “A. 15-lead”, “B. Late potentials”, “C. P-wave signal averaging” “D. ACI-TIPI”, and “E. Exercise Testing.” The answers selected by the customer to this question may be used to identify resting ECG system options to recommend to the customer or to identify the resting ECG system that is most appropriate for the customer.

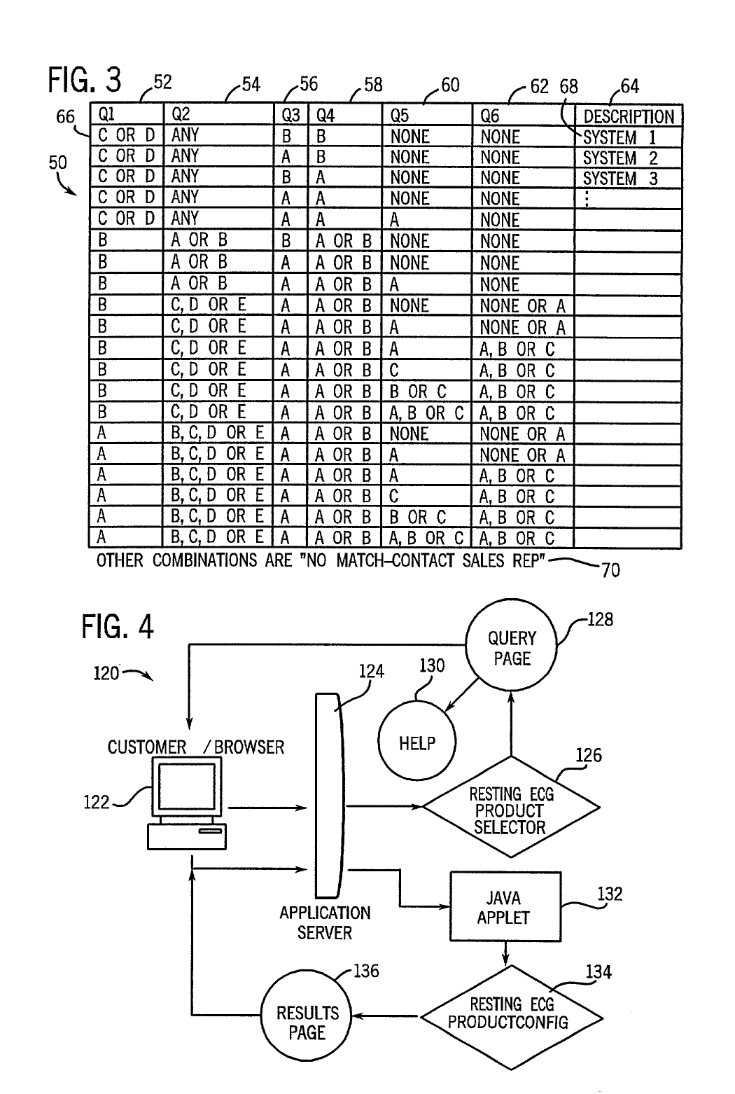

Referring generally to FIG. 3, an exemplary logic template 50 is provided. The logic template 50 correlates combinations of answers to the questions to resting ECG systems. A resting ECG system is recommended to the customer if the combination of answers provided by the customer correspond to a combination of answers in the template 50 that have a resting ECG associated with them.

In the illustrated embodiment, answers to each of the questions listed in FIG. 2 are shown in separate columns. Answers to the first question 38 are provided in a first column 52. Answers to the second question 40 are provided in a second column 54. Answers to the third question 42 are provided in a third column 56. Answers to the fourth question 44 are provided in a fourth column 58. Answers to the fifth question 46 are provided in a fifth column 60. Answers to the sixth question 48 are provided in a sixth column 62. Additionally, there is a column 64 for resting ECG systems 68. Each row comprises a combination of answers to the questions and a resting ECG system that is recommended to a customer when that customer answers the questions according to the combination shown. For example, in the first row 66, the resting ECG system 68 is identified as “SYSTEM 1.” SYSTEM 1 is recommended when the customer: selects answer “C. Clinic” or “D. Private Practice” to the first question 38 (What is your clinical setting?); selects any of the answers to the second question 40 (How many ECG test will you perform on average during a month?); selects answer “B. No” to the third question 42 (Do you require interpretation?); selects answer B to the fourth question 44 (Do you require fall size paper?) and selects none of the answers to the fifth question 46 (What communication options do you require?) or the sixth question 48 (What other ECG applications do you require?). The rows following the first row 66 correspond to other combinations of answers that correspond to recommended resting ECG systems.

Not every resting ECG system supplied by a supplier may be one of the available recommended resting ECG systems. The resting ECG systems selected as recommended resting ECG systems may be selected for a number of reasons, such as their popularity with customers based on previous sales, ease of configuration with options, etc. In the illustrated embodiment, a spillover row 70 is shown that corresponds to the situation where the combination of answers provided by the customer does not match any of the answers associated with a recommended resting ECG system. In this situation, the supplier may not recommend a specific resting ECG system. Rather, the supplier may inform the customer to contact a sales representative to identify the best resting ECG system for the customer's resting ECG system needs.

Referring generally to FIG. 4, a system 120 is illustrated that enables a resting ECG system supplier to provide a customer with a recommended resting ECG system. In the illustrated embodiment, a customer may use a computer 122, or other browsing device to access the system 120 over a network, such as the Internet. The system 120 utilizes an application server 124, a resting ECG product selector file 126, a query page 128, a help page 130, a Java applet 132, a resting ECG product configuration file 134 and a results page 136 to provide a customer with a recommended resting ECG system. The application server 124 is used to route information around system 120. The application server may comprise a program, such as a Java class. The resting ECG product selector file 126 holds the data that is used to populate the query page 128 to be supplied to the customer. The query page 128 contains the question designed to enable a resting ECG system supplier to recommend a resting ECG system to meet the customer's needs, or at least provide a basis for discussion between the supplier and the customer. The help page 130 is linked to the query page 128 and contains additional information designed to assist a customer in answering at least one of the questions.

The Java applet 132 forms part of the logic functions illustrated in FIG. 3. The Java applet 132 compares the answers in the completed query page 128 to data in the resting ECG product configuration file 134. The resting ECG product configuration file contains a set of data for a plurality of resting ECG systems that correspond to predicted responses to the questions in the resting ECG product selector file. All possible combinations of answers to the plurality of questions may be provided with an associated, or recommended, resting ECG system. Alternatively, some, but not all, of the possible combinations may be provided with a corresponding, or recommended, resting ECG system. This smaller set of combinations may be based on a number of factors. For example, the resting ECG product configuration file 134 may be written so that a recommendation is made only for the most commonly sold configurations of resting ECG systems. If the Java applet 132 finds a match between the customer's response and a predicted response, the resting ECG system information corresponding to the predicted response is provided to the customer via the results page 136. If there is no match, an advisory to contact a sales representative may be provided to the customer.

In the illustrated embodiment, the resting ECG product selector file 126 and the resting ECG product configuration file 134 are XML files. XML is a method for putting structured data in a text file. XML is powerful because it maintains the separation of the user interface from structured data. HTML specifies how to display data in a browser, but XML defines the content. For example, in HTML tags are used to tell the browser to display data as bold or italic. In XML, style sheets are employed to present the data in a browser. XML separates the data from the presentation and processing, enabling data to be displayed and processed differently by applying different style sheets and applications.

As will be appreciated by those skilled in the art, XML is a meta-markup language that provides a format for describing structured data. This facilitates more precise declarations of content and more meaningful search results across multiple platforms. An unlimited set of tags may be defined in XML. As noted above, while HTML tags may be used to display a word in bold or italic, for example, XML provides a framework for tagging structured data. An XML element can declare its associated data to be a price, a tax, a title, or any other desired data. As XML tags are adopted, there will be a corresponding ability to search for and manipulate data regardless of the applications within which it is found. Once data has been located, it can be delivered over a network and presented in a browser in any number of ways, or it can be handed off to other applications for further processing and viewing.

XML is a subset of the Standard Generalized Markup Language (SGML) that is optimized for delivery over the Web. XML provides a data standard that can encode the content, semantics, and schemata for a wide variety of cases ranging from simple to complex, and which may be used to markup the following: an ordinary document; a structured record, such as an appointment book or purchase order; an object with data and methods, such as the persistent form of a Java object; a data record, such as the result set of a query, meta content of a site; graphical presentations; standard schema entities and types; and all links between entities and types. Once the data is on the client's desktop it can be manipulated, edited, and presented in multiple views, without returning to the server. Servers may then become more scalable, due to lower computational and bandwidth loads. Also, because data is exchanged in the XML format, it can be easily merged from different sources. In the illustrated embodiment, the resting ECG product selector file is an XML file. However, resting ECG product selector file 126 may be defined by other file or application types, including a different markup language, such as HTML.

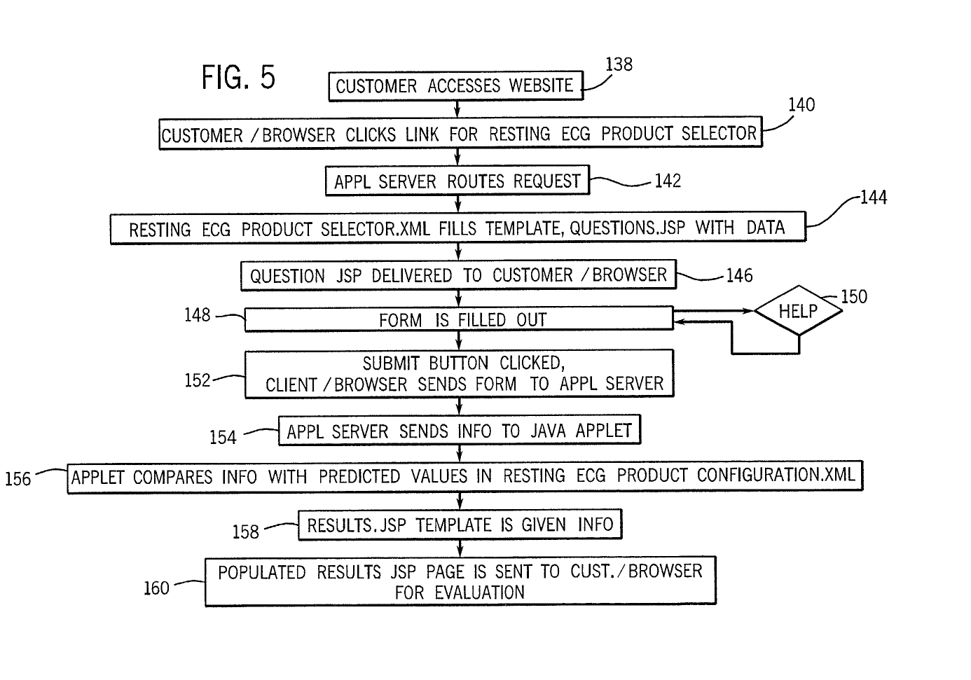

Referring generally to FIG. 5, an exemplary process by which a customer may be provided with information for a recommended resting ECG system is illustrated. Initially, a customer or client accesses a site, as referenced by step 138. The site may provide a resting ECG system supplier home page, a resting ECG system specification page, a product selector page, or some other page of interest to a customer or client interested in purchasing a supplier's resting ECG systems, resting ECG system components, software, etc. Initially, the client or customer activates a link on the page to enter a resting ECG system product selector, as referenced by step 140. An application server routes the request to enter the resting ECG system product selector to a resting ECG System product selector file, as referenced by step 142. In an exemplary embodiment, the resting ECG product selector file is an XML file. The resting ECG System product selector file contains the data representing the questions to ask a customer to narrow the choice of a resting ECG system, component, or software to a recommended resting ECG system, component, or software. The resting ECG System product selector file fills the query page template with the data, e.g., questions, as referenced by step 144. In an exemplary embodiment, the query page is a Java script file. The query page is sent to the customer browser for completion, as referenced by step 146.

The customer then completes the query page, as referenced by step 148. If a customer would like additional information to help in choosing an answer, the client may activate a link to a help file, as referenced by step 150. When the link is activated, a help page 130 is presented to the customer. In this embodiment, the help page 130 provides additional information specific to each question on the query page. The additional information is designed to assist the customer answer the questions. After obtaining the information, the customer may then return to the query page and continue answering questions. When the customer has completed answering the questions, the completed query page is sent back to the application server by activating a virtual button on the query page, as referenced by step 152.

In the exemplary process, the application server couples the completed query page 128 to a Java applet 132, as referenced by step 154. The Java applet 132 compares the information provided by the customer in their answers to the questions in the query page to predicted responses to the questions contained within the product configuration file 134, as referenced by step 156. If a match is found between the customer's response to the questions and the predicted responses to the questions, the Results page 136 is filled with data describing the recommended resting ECG system, component, or software, that corresponds to the predicted responses to the questions, as referenced by step 158. The Results page 136 is then provided to the customer for viewing and/or evaluation, as referenced by step 160.

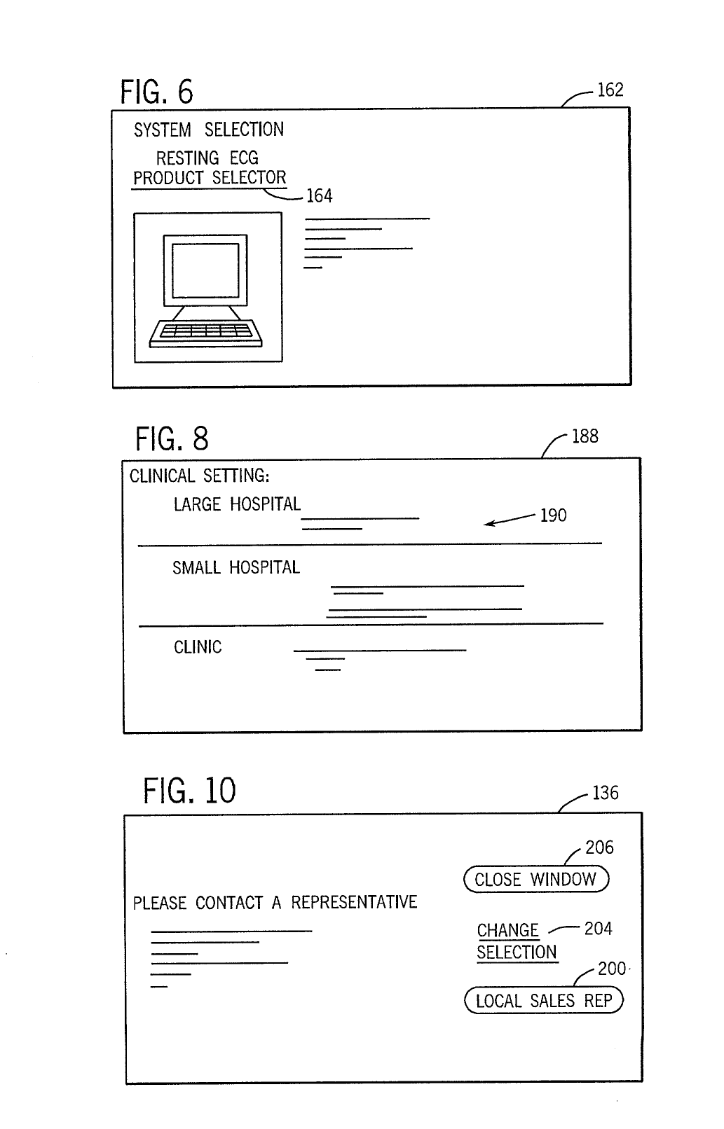

Referring generally to FIG. 6, a page 162 for a resting ECG system supplier is illustrated. The page 162 may contain information about the suppliers resting ECG systems, components or software. In addition, the page 162 contains a link 164 to a resting ECG product selector.

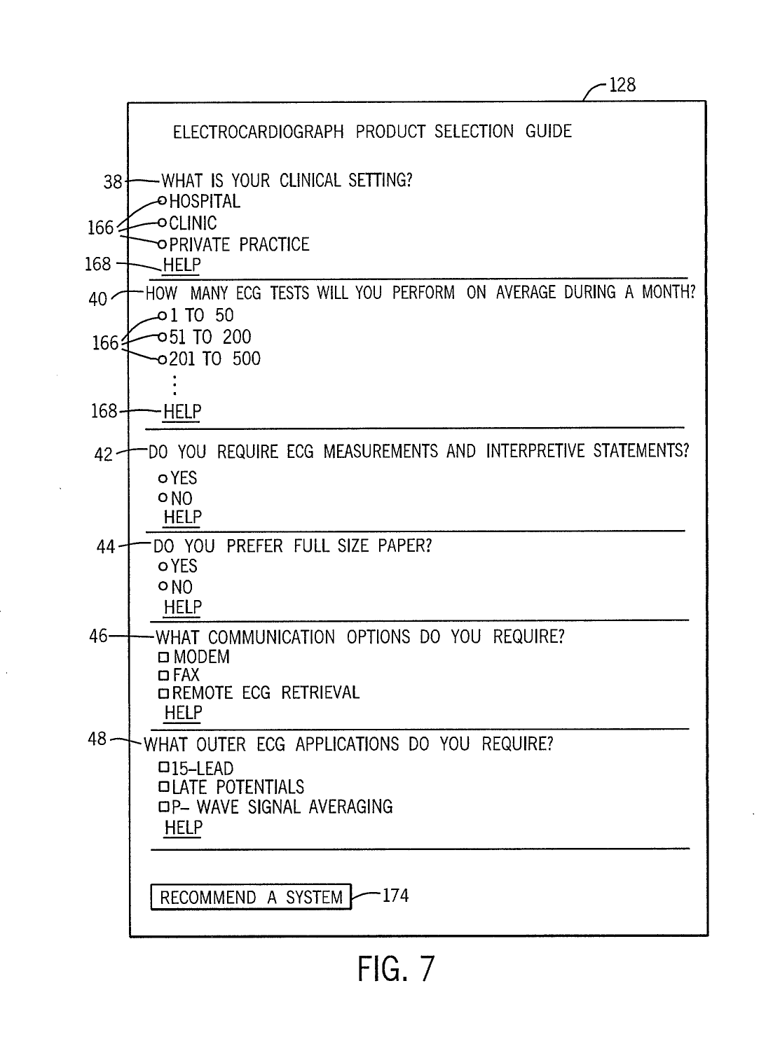

Referring generally to FIG. 7, when a customer activates the link 164, system 120 operates to provide the customer browser 122 with a query page 128. In the illustrated embodiment, the page presents the series of questions illustrated in FIG. 2 to the customer. Each question is answered by selecting one or more of the choice registers 184. Some questions may have mutually exclusive answers while others may allow multiple answers. The choice registers 184 may be selected by placing the cursor over a choice register 184 and clicking with a mouse virtual button.

In addition, each question may have a link to the help file 130. When the link is activated a help window 188 is opened, as best illustrated in FIG. 8. In the illustrated embodiment, the link 186 after the question, “What is your clinical setting?” has been activated. Help window 188 contains text 190 describing the various clinical settings. However, help window 188 and help file 130 may be populated with any type of information that may be designed to assist a customer in answering any of the questions provided to the customer.

Referring again to FIG. 7, the customer may activate a virtual button 194 when the customer has made their selections and would like a resting ECG system to be recommended to the customer. Activating the virtual virtual button 194 causes system 120 to operate to provide the customer with a recommended resting ECG system, component or software.

Referring generally to FIG. 9, system 120 provides the recommendation for a resting ECG system, component, and/or software on a results page 136. The results page 136 provides the customer with a recommended system 196 and recommended options 198. The customer may then contact the supplier to further discuss the purchase of the recommended resting ECG system. For example, the customer may want to discuss in more detail the specifications of the recommended resting ECG system or discuss pricing or financing. The customer may want to discuss the purchase of other resting ECG systems. Consequently, results page 136 may have a virtual button 200 that is operable to activate a program to identify a supplier sales representative located near the customer. Alternatively, the customer may want to proceed directly to the purchase of the recommended resting ECG system. Thus, the results page 136 may also have a purchasing link 202 to initiate a purchasing program to enable a customer purchase a resting ECG system, component, and/or software from the page. The results page 136 may also have a link 204 to enable a customer perform another product selection with the resting ECG product selector.

As best illustrated in FIG. 10, if there is no resting ECG system, component, or software that matches the customer's responses to the questions the results page 136 may inform the customer that no resting ECG system, component, or software may be recommended based on the customer's responses to the questions. The customer may be informed to contact a resting ECG system supplier's sales representative. Therefore, this embodiment of results page 136 also may have a virtual button 200 that is operable to activate a program to identify the supplier's sales representative nearest to the customer. The results page 136 also may have a virtual button 206 to close the browser or to close the product selector.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention is to cover all modifications, equivalents, and alternatives falling within the spirit and scope of the invention as defined by the following appended claims.

1. A computer system coupled to a network to enable a resting electrocardiogram (ECG) system supplier to provide a customer with information relating to a recommended resting ECG system, the computer system comprising:
an application server to direct a query page to the customer via the network, the query page comprising a plurality of questions and a plurality of answer choices for each question, wherein a customer's selection of an answer choice to at least one of the plurality of questions enables the computer system to determine a recommended resting ECG system, further wherein at least one of the plurality of questions establishes whether resting ECG system with a capability of interpreting a patient's heart condition is to be recommended;
a comparison program to receive a completed query page from the customer and compare the customer's answer choices in the completed query page to information stored in the computer system to determine a recommended resting ECG system; and
a server to provide a results page to the customer via the network, the results page providing the customer with the recommended resting ECG system.

2. The system as recited in claim 1, wherein at least one of the plurality of questions is designed to determine a frequency range that the customer may perform resting ECG examinations with a resting ECG system.

3. The system as recited in claim 1, wherein at least one of the plurality of questions is designed to determine resting ECG capabilities that the customer may desire.

4. The system as recited in claim 3, wherein a resting ECG capability is P-Wave signal averaging.

5. The system as recited in claim 1, wherein at least one of the plurality of questions is designed to identify a specific sales entity from among a plurality of sales entities to conduct business with the customer.

6. The system as recited in claim 1, wherein the application server comprises a Java class.

7. The system as recited in claim 1, wherein the comparison program comprises a Java applet.

8. The system as recited in claim 1, further comprising a product selector file, wherein the product selector file contains the plurality of questions for supplying the query page.

9. The system as recited in claim 8, wherein the product selector file is written in extensible markup language (XML).

10. The system as recited in claim 1, wherein the query page is written in Java script.

11. The system as recited in claim 1, wherein the query page comprises a link to a help page, wherein the help page provides information to assist a customer answer at least one of the plurality of questions.

12. The system as recited in claim 1, wherein each question has an associated link to a help page, wherein the help page provides information to assist a customer answer each of the plurality of questions.

13. The system as recited in claim 1, wherein the information stored in the computer system is stored in a product configuration file, wherein the product configuration file contains data on specific configurations of resting ECG systems.

14. The system as recited in claim 13, wherein the specific configurations of resting ECG systems are based on sales history of the specific configurations of resting ECG systems.

15. The system as recited in claim 14, wherein a specific configuration of a resting ECG system comprises software packages.

16. The system as recited in claim 13, wherein the product selector file is written in extensible markup language (XML).

17. The system as recited in claim 13, wherein the product selector file populates the results page with a specific resting ECG system configuration that matches the customer's responses in the completed query page..

18. The system as recited in claim 1, wherein the results page is written in Java script.

19. A computer system coupled to a network to enable a resting electrocardiograph (ECG) system supplier to provide a customer with a recommended resting ECG system over the network, the computer system comprising:
an application server coupled to a network, the application server directing a customer to files stored in the computer system;
a product selector file written in a markup language and stored in the computer system, the product selector file defining a plurality of questions designed to elicit data from a customer to determine a resting ECG system to recommend to the customer from among a plurality of resting ECG systems, wherein the product selector file provides the plurality of questions to a query page for delivery to a customer;
a program that operates to determine a recommended resting ECG system for the customer by comparing data provided by the customer via the plurality of questions to resting ECG system data stored in the computer system; and
a product configuration file written in a markup language and stored in the computer system, the product configuration file holding the resting ECG system data used by the program, wherein the product configuration file provides information relating to a recommended resting ECG system to a results page for delivery to the customer.

20. The system as recited in claim 19, wherein at least one of the plurality of questions is designed to determine whether a customer desires computer software to operate with the resting ECG system to analyze results from a patient's resting ECG examination to determine the patient's heart condition.

21. The system as recited in claim 19, wherein at least one of the plurality of questions is designed to determine a frequency range that the customer may perform resting ECG examinations with the resting ECG system.

22. The system as recited in claim 19, wherein at least one of the plurality of questions is designed to determine resting ECG capabilities that the customer may desire.

23. The system as recited in claim 22, wherein a resting ECG capability is P-Wave signal averaging.

24. The system as recited in claim 19, wherein at least one of the plurality of questions is designed to identify a specific sales entity from among a plurality of sales entities to conduct business with the customer.

25. The system as recited in claim 19, wherein the product selector file is written in extensible markup language (XML).

26. The system as recited in claim 19, wherein the product configuration file is written in extensible markup language (XML).

27. The system as recited in claim 19, wherein each question is a multiple-choice question.

28. The system as recited in claim 19, further comprising a help file written in a markup language and containing information regarding each choice in at least one multiple-choice question.

29. The system as recited in claim 28, wherein the help file is written in hypertext markup language (HTML).

30. The system as recited in claim 19, wherein the application server is a Java class.

31. The system as recited in claim 19, wherein the program is a Java applet.

32. The system as recited in claim 19, wherein the query page is written in a Java script language.

33. The system as recited in claim 19, wherein the results page is written in a Java script language.

34. The system as recited in claim 19, wherein a recommended resting ECG system comprises software.

35. A method of utilizing a computer system coupled to a network to assist a customer to configure a resting electrocardiograph (ECG) system from among a plurality of resting ECG systems, components and software, the method comprising the acts of:
routing a request for assistance from a customer to a product selector file written in extensible markup language (XML), wherein the product selector file fills a template with questions stored in the product selector file;
delivering the template over the network to a customer;
receiving a completed template from the customer; and
determining a recommended resting ECG system configuration by comparing customer data derived from the completed template to supplier data stored in the computer system in a product configuration file written in XML, wherein the product configuration file fills a results page with the recommended resting ECG system configuration for delivery to the customer over the network.

36. The method as recited in claim 35, wherein routing comprises activating a link in a page to an application server.

37. The method as recited in claim 36, wherein the application server routes the request to the product selector file.

38. The method as recited in claim 35, wherein the template is a Java script file.

39. The method as recited in claim 35, wherein the results page is a Java script file.

40. A method of utilizing a computer system to configure a recommended resting electrocardiograph (ECG) system from among a plurality of resting ECG systems, components and software, the method comprising the acts of:
connecting a customer communication system to a computer system provided by a resting ECG system supplier;
routing a request for assistance from the customer to a product selector file written in extensible markup language (XML), wherein the product selector file fills a template with questions stored in the product selector file;
delivering the template to a customer;
completing the template with the customer communication system and transmitting it to the computer system;
receiving a completed template from the customer; and
determining a recommended resting ECG system and configuration by comparing customer data derived from the completed template to supplier data stored in the computer system in a product configuration file written in XML, wherein the product configuration file fills a results page with the recommended resting ECG system configuration for delivery to the customer communication system.

41. The method as recited in claim 40, wherein the customer communication system is a second computer system having an interface coupled to the Internet.

42. The method as recited in claim 40, wherein the supplier data comprises data for a plurality of resting ECG system configurations of components and software.

43. The method as recited in claim 40, wherein determining comprises using a program to compare the customer data to the plurality of resting ECG system configurations of components and software.

44. The method as recited in claim as recited in claim 40, wherein the product configuration file provides the results page with the data for a specific resting ECG system configuration of components and software when the program identifies a specific resting ECG system configuration that matches the customer data.
